(19) United States
(12) Patent Application Publication
(10) Pub. No.: US 2019/0117280 A1
(43) Pub. Date: Apr. 25, 2019

(54) THREADED SPINAL ROD REDUCER

(71) Applicant: SPINE WAVE, INC., Shelton, CT (US)

(72) Inventors: Eugene Avidano, Stratford, CT (US); Dylan Freund, Southbury, CT (US); Mark Serokosz, New Fairfield, CT (US)

(21) Appl. No.: 16/129,957

(22) Filed: Sep. 13, 2018

Related U.S. Application Data
(60) Provisional application No. 62/575,140, filed on Oct. 20, 2017.

(51) Int. Cl. A61B 17/70; A61B 17/88

(52) U.S. Cl. CPC A61B 17/7086; A61B 17/7085; A61B 17/88

(57) ABSTRACT

A spinal rod reducer for reducing a rod into a rod-receiving opening of a bone anchor comprises an elongate tubular body having a lumen extending therethrough and a pair of spaced flexible anchor attachment members at the distal end of the tubular body. A threaded rod reduction member having a rod contacting surface is supported for axial movement within the lumen. A locking sleeve for slidable engagement with the attachment members to fixedly secure the attachment members to the bone anchor is supported for axial movement on the tubular body. A threaded rotatable reduction knob is threadably engaged with the rod reduction member rotation of which causes the rod reduction member with the rod contacting surface to move axially distally toward the bone anchor. Movement of the locking sleeve is independent of the movement of the rod reduction member.

### CROSS-REFERENCES TO RELATED APPLICATIONS

This application claims the benefit of U.S. Provisional Patent Application No. 62/575,140, filed Oct. 20, 2017, which is incorporated herein by reference in its entirety.

### FIELD OF THE INVENTION

The subject invention relates generally to the field of spinal surgery instruments, and more particularly to a threaded rod reducer for reducing a spinal fixation rod into a bone anchor.

### BACKGROUND OF THE INVENTION

Techniques and systems have been developed for correcting and stabilizing injuries to or malformation of the spine. In one type of system, an elongated member such as a bendable spinal fixation rod is disposed longitudinally along a length of the spine, spanning two or more vertebral levels. In certain applications, the rod is bent to correspond to the normal curvature of the spine in the particular region being instrumented, such as the normal kyphotic curvature of the thoracic region or the lordotic curvature of the lumbar region. In accordance with such a system, the rod is engaged to various vertebrae along a length of the spinal column by way of a number of anchor devices that utilize a variety of fixation elements configured to engage specific portions of the vertebra and other bones. For instance, one such fixation element is a hook that is configured to engage the laminae of the vertebra. Another very prevalent fixation element is a pedicle screw that can be threaded into various parts of the vertebrae or other bones. Such pedicle screws may be monoaxial or polyaxial, such as the multi-axial screws disclosed in commonly assigned U.S. Pat. No. 8,162,898, entitled “Multi-Axial Fixation Assembly”, issued to Potash et al. on Apr. 24, 2012, the disclosure of which is incorporated herein by reference.

Once bone anchors, such as pedicle screws are anchored in the vertebrae, a connecting rod must be nestled or seated within a rod-receiving opening or the slot of each pedicle screw. This approach is often challenging in part because the rod introduction site is not readily visible or because there are no suitable tactile indicators that the rod is properly seated in every bone screw assembly. In order to ensure proper placement of the rod, a rod reducer or persuader may be mounted on one or more of the pedicle screws to provide a sufficient force to the rod to properly and fully urge the rod into the rod-receiving opening.

There are a variety of known rod reduction instruments that are used by surgeons to provide the desired forces required to appropriately reduce spinal fixation rods into bone anchors during surgical procedures. Nevertheless, there is interest in a rod reducer that has features that are simple and easy to use and that may result in less time for the surgeon to complete the rod reduction procedure.

### SUMMARY OF THE INVENTION

It is an object of the invention to provide an improved rod reducer for use during surgery, such as spinal surgery. It is a more particular object of the invention to provide a threaded rod reducer that allows the user to reduce spinal fixation rods into bone anchors, such as pedicle screws, in a simple, less time-consuming procedure.

### DESCRIPTION OF THE FIGURES

FIG. 1 is a side elevation view of a threaded rod reducer for use during spinal surgery in accordance with one arrangement of the present invention.

FIG. 2 is a longitudinal cross-sectional view of the rod reducer as seen along viewing lines II-II of FIG. 1.

FIG. 3 is an exploded view of the rod reducer of FIG. 1.

FIG. 4 is a top plan view of the rod reducer of FIG. 1 with the locking sleeve located in a first position allowing attachment to a bone anchor,

FIG. 5 is a view of the rod reducer of FIG. 4 with the locking sleeve moved to a second position for secure attachment to a bone anchor.

FIGS. 6A-G show a sequence of steps for use of the subject rod reducer in a spinal surgical procedure.

### DESCRIPTION OF THE EMBODIMENTS

For the purposes of promoting and understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

Traditional polyaxial pedicle screws comprise an elongate shaft threaded at one end and a head, typically having a spherical surface, at the other end. A yoke having a U-shaped opening for receiving a spinal fixation rod is typically pre-assembled to the screw head in a manner to allow articulating movement of the yoke relative to the threaded shaft. In a modular pedicle screw construction, the yoke is configured to be articulatingly attached to the screw head subsequent to the threaded installation of the threaded shaft into a pedicle. The rod reducer of the subject invention is applicable for use with both a traditional polyaxial pedicle screw after threaded installation into a pedicle or with a modular polyaxial pedicle screw after in situ attachment of the yoke to the pedicle screw head.

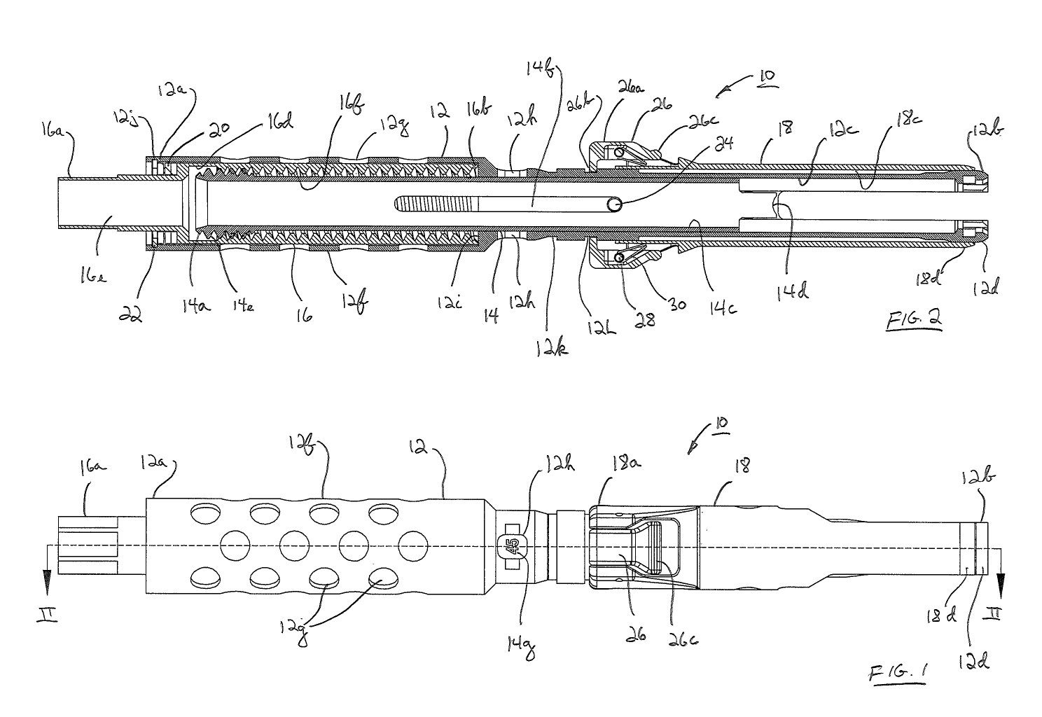

Turning now to FIGS. 1-3 a spinal rod reducer 10 in accordance with a particular arrangement of the subject invention is shown. Rod reducer 10 comprises an elongate tubular body 12, a threaded rod reduction member 14, a threaded rotatable reduction knob 16, and a locking sleeve 18. Tubular body 12 has a proximal end 12a and a distal end 12b and a lumen 12c extending therethrough. A pair of spaced, flexible anchor attachment members 12d is disposed adjacent distal end 12b of tubular body 12, anchor attachment members 12d defining therebetween a slot 12e opening at the distal end 12b and extending for a length proximally. Attachment members 12d are configured to engage a bone anchor, such as a pedicle screw, in a manner to align slot 12e with the U-shaped rod receiving opening of the bone anchor. Tubular body 12 has an enlarged portion 12f adjacent proximal end 12a, enlarged portion 12f serving as a handle for rod reducer 10 and including in a particular arrangement a plurality of openings 12g to assist the user in grasping rod reducer 10. Between proximal end 12a and distal end 12b tubular body 12 has at least one window 12h extending therethrough in communication with lumen 12c. In a particular arrangement, a second window 12h is provided on the opposite side of tubular body 12. The purpose of windows 12h will be described hereinafter.

Threaded rod reduction member 14, as illustrated in FIGS. 1-3, has a proximal end 14a and a distal end 14b, and is supported by tubular body 12 for axial movement within tubular body lumen 12c, as will be described. In a particular arrangement, rod reduction member 14 is a generally cylindrical inner tube having a central lumen 14c. Distal end 14b of rod reduction member 14 includes a rod contacting surface 14d for engaging a spinal fixation rod to urge such rod into the rod-receiving opening of the bone anchor upon movement of rod reduction member 14 distally, as will be described. Rod contacting surface 14d may include a curved indentation, as shown in FIG. 3, to substantially conform to the curvature of the spinal rod to assist in the reduction process. In the particular arrangement where rod reduction member is tubular, rod contacting surface 14d may be defined by a pair of diametrically spaced portions that together engage a spinal fixation rod at two axially spaced locations along the rod axis during rod reduction.

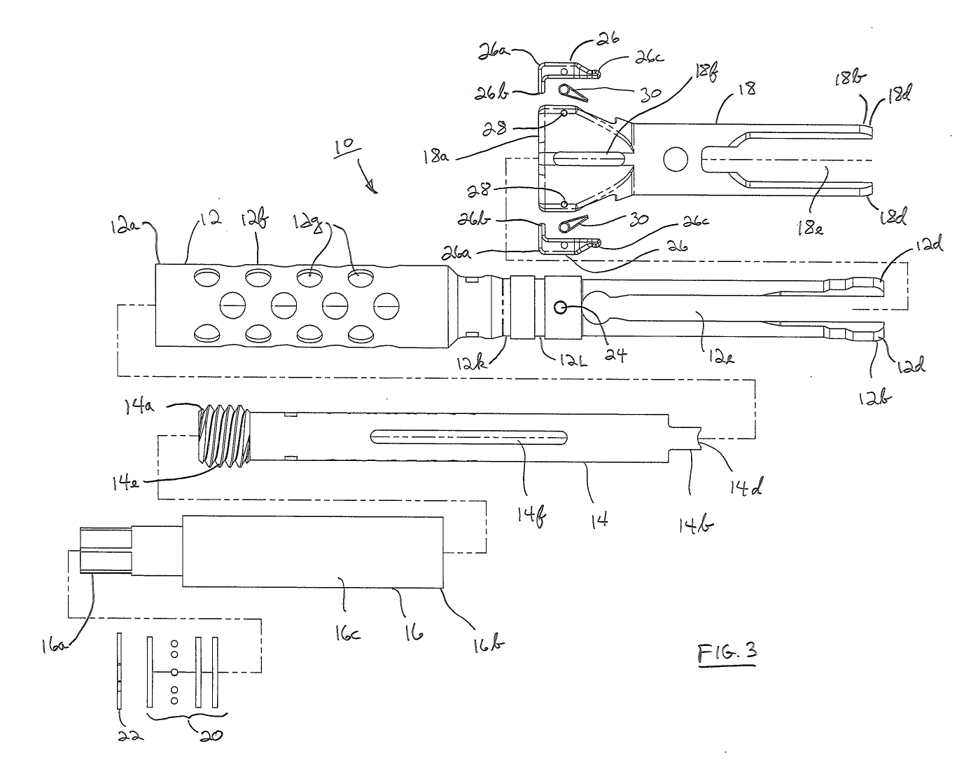

As illustrated particularly in FIGS. 2-3, rod reduction member 14 includes exterior threads 14e at proximal end 14a. Rod reduction member 14 includes an elongated slot 14f extending therethrough in communication with lumen 14c, the purpose of which will be described. Rod reduction member 14 includes thereon a first series of spaced indicia 14g that are axially spaced to individually align with at least one window 12h upon axial movement of rod reduction member 14 within lumen 12c of tubular body 12. Each individual indicia 14g is representative of a distance a spinal fixation rod is to be reduced into the rod-reducing opening of the bone anchor, as will be described. In a particular arrangement, rod reduction member 14 may include a second series of such spaced indicia 14g on the opposite side, such second spaced indicia 14g being aligned axially with the first series of indicia 14g and being representative of the same distances as the first series of indicia 14g. The second series of spaced indicia 14g are also axially spaced to individually align with at the second window 12h upon axial movement of rod reduction member 14 within lumen 12c of tubular body 12. As such, the same representative distance would be displayed through both windows 12h.

Threaded rotatable reduction knob 16 as shown in FIGS. 1-3 has a proximal end 16a and a distal end 16b. Reduction knob 16 in a particular arrangement is generally cylindrical having an outer surface 16c, an inner surface 16d and a central lumen 16e extending therethrough. Inner surface 16d comprises interior threads 16f configured to be in threaded engagement with exterior threads 14e of rod reduction member 14, as depicted in FIG. 2. Reduction knob 16 is further configured to reside within enlarged portion 12f of tubular body 12 such that distal end 16b engages a transverse shoulder 12i within lumen 12c of tubular body 12, as illustrated in FIG. 2. This prevents reduction knob 16 from moving axially distally relative to tubular body 12. Adjacent proximal end 16a, reduction knob 16 is contained within tubular body 12 by a thrust bearing 20 and a locking clip 22. Locking clip 22 is supported within an interior groove 12j at the proximal end 12a of tubular body 12. Locking clip 22 prevents reduction knob 16 from moving axially proximally relative to tubular body 12. Thrust bearing 20 allows reduction knob 16 to rotate within lumen 12c of tubular body 12. Therefore, while reduction knob 16 can rotate while not moving axially, rod reduction member 14 is keyed to tubular body 12, as will be described, to move axially but not rotationally. As such, as a result of the threaded connection between threads 14e/16f of rod reduction member and reduction knob 16, respectively, rotation of reduction knob 16 will axially move rod reduction member 14 within tubular body 12. Reduction knob 16 includes a tool engagement portion 16g extending outwardly beyond proximal end 12a of tubular body 12, tool engagement portion 16g being configured to mate with a tool (not shown) for applying a rotational force to reduction knob 16.

Details of locking sleeve 18 are now described with respect to FIGS. 2-3. Locking sleeve 18 has a proximal end 18a, a distal end 18b and a lumen 18c extending therethrough. A pair of spaced locking members 18d is disposed adjacent distal end 18b of locking sleeve 18, locking members 18d defining therebetween a channel 18e opening at the distal end 18b and extending for a length proximally. Locking sleeve 18 is slidably supported for axial but not rotational movement on distal end 12b of tubular body 12. Such axial movement of locking sleeve 18 is independent of any movement of rod reduction member 14. During such axial movement locking sleeve channel 18e is aligned with tubular body slot 12e as locking sleeve 18 is axially moved from a first position to a second position, as will be described. Locking sleeve 18 includes an elongated slot 18f extending therethrough in communication with lumen 18c. A pair of pins 24, supported by tubular body 12 and extending generally diametrically oppositely outwardly from tubular body 12, communicates with rod reduction member lumen 14c and reduction knob lumen 16c. Pins 24 are received in locking sleeve elongated slot 18f, allowing a limited amount of axial movement of locking sleeve 18 relative to tubular body 12 while preventing relative rotational movement therebetween. Similarly pins 24 are received in rod reduction member elongated slot 14f, allowing a limited amount of axial movement of rod reduction member 14 relative to tubular body 12 while preventing relative rotational movement therebetween. As such, both locking sleeve 18 and rod reduction member 14 are keyed to tubular body by pins 24.

Referring still to FIGS. 1-3, locking sleeve 18 includes a pair of locking elements 26 supported generally diametrically oppositely at the proximal end 18a of locking sleeve 18. In a particular arrangement, each locking element 26 comprises a spring-loaded latch 26a that is supported by a pin 28 for pivotal movement thereon. A torsion spring 30 may be provided in association with each pin 28 and latch 26a to bias each latch 26a toward tubular body 12, as will be described. Each latch 26a has at one end a contact portion 26b configured to engage a portion of tubular body 12, such as cavities 12k and 12L formed into the wall of tubular body 12, as shown in FIGS. 2 and 3. Cavities 12k and 12L in a particular arrangement are axially spaced grooves formed circumferentially around tubular body 12. Cavity 12k is engaged by spring latch contact portion 26b when locking sleeve 18 is in the first position, and cavity 12L is engaged by spring latch contact portion 26b when locking sleeve 18 is in the second position, as will be described. The other end of each latch 26a includes a projecting button 26c that may be manually depressed in a manner to overcome the bias force provided by torsion spring 30. While a pair of locking elements 26 is described it should be appreciated that a single locking element 26 or more than two locking elements 26 may be used.

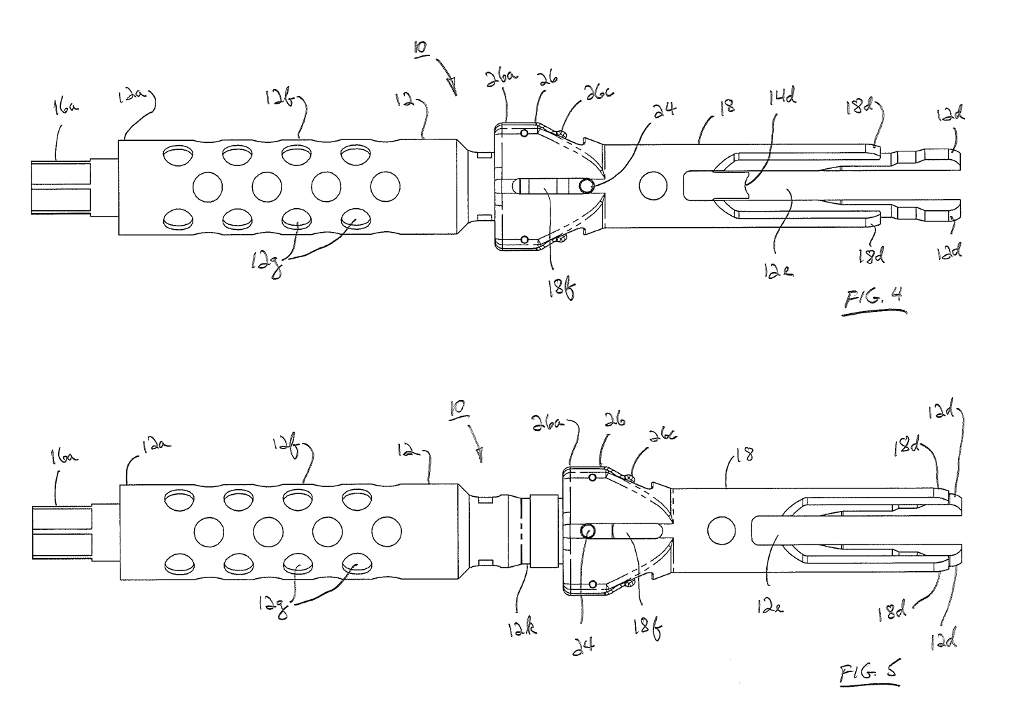

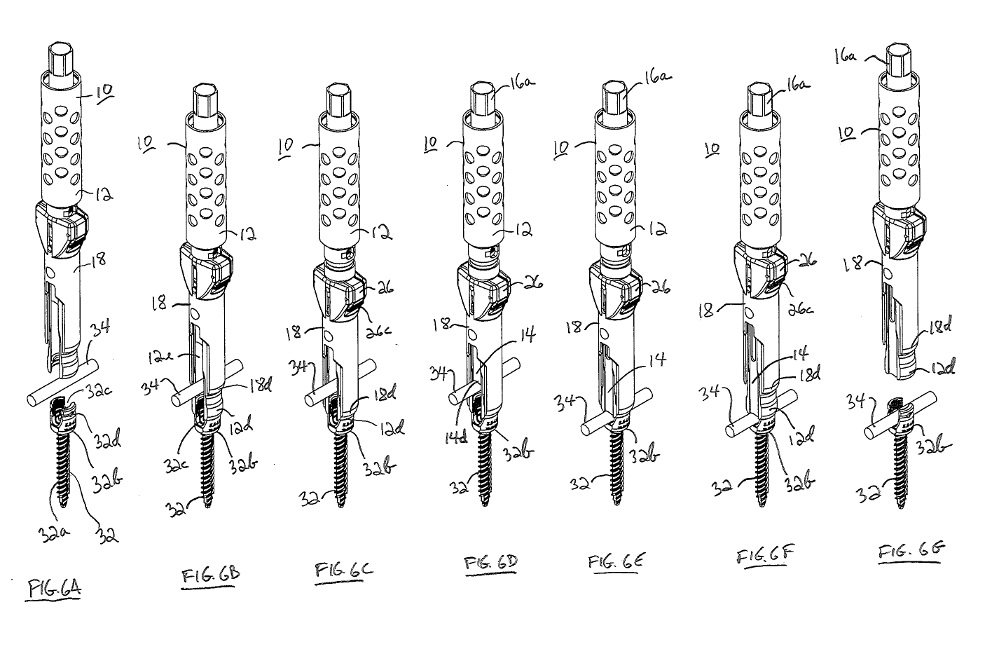

As shown in FIG. 4, locking sleeve 18 is shown in the first position relative to tubular body 12. In this first position, spring latch contact portions 26b are biased into engagement with tubular body cavities 12k by torsion spring 30. As such, locking sleeve 18 is releasably secured in this fixed axial first position relative to tubular body 12. In this first position, locking sleeve locking members 18d are spaced axially proximally of and thereby exert minimal radial constraints on tubular body anchor attachment members 12d, allowing anchor attachment members 12d to flexibly snap onto a bone anchor, such as the yoke of a pedicle screw. In this first position, pin 24 is situated at the distalmost location within locking sleeve elongated slot 18f. Depression of spring latch buttons 26c in a manner to overcome the bias force of torsion spring 30 allows contact portions 26b to be pivotally released from cavities 12k thereby allowing locking sleeve 18 to move axially distally to the second position, as shown in FIG. 5. In this second position, locking sleeve locking members 18d substantially cover and thereby radially constrain movement of anchor members 12d to form a secure attachment between anchor members 12d and the bone anchor. In the second position spring latch contact portions 26b are moved to cavities 12L (as shown in FIG. 2), thereby releasably securing locking sleeve 18 relative to tubular body 12 under the bias provided by torsion spring 30. In this second position, pin 24 is situated at the proximalmost location within locking sleeve elongated slot 18f, as depicted in FIG. 5.

Having described the details of threaded rod reducer 10, the use thereof in a surgical procedure is now described. Once a desired number of bone anchors, such as pedicle screws, are suitably attached to vertebrae of a spine the spinal fixation rod must be nestled or seated within the U-shaped rod-receiving opening of the pedicle screw yoke, as noted hereinabove. The rod reducer 10 of the subject invention may be attached to the pedicle screw yoke either before the fixation rod is introduced to the surgical site or after the fixation rod is placed into the pedicle screw yokes but not fully reduced. In the first instance of use, the arms of the yoke defining the U-shaped opening may serve as a guide to initially introduce the fixation rod, while in the second instance of use slot 12e of the rod reducer tubular body 12 may serve as the rod introduction guide.

The sequence of the steps of using rod reducer 10 in a spinal surgical procedure, such as in an open or mini open approach, is illustrated FIGS. 6A-G. FIG. 6A shows a pedicle screw 32 having a threaded shaft 32a and a yoke 32b having a U-shaped rod-receiving opening 32c. Yoke 32b may have an external groove 32d for releasable attachment to reducer 10. Pedicle screws 32 may be monoaxial or polyaxial, such as those described hereinabove. An elongated spinal fixation rod 34 is shown in position slightly above pedicle screw 32 with threaded rod reducer 10 positioned above rod 34 in the loading position and ready for reducing rod 34 into rod-receiving opening 32c. At this time, locking sleeve 18 of rod reducer 10 is in the first position as shown in FIG. 4 whereby anchor attachment members 12b are not constrained and are capable of flexing. In FIG. 6B rod reducer 10 is flexibly attached to pedicle screw 32 by snapping flexible anchor attachment members 12d into groove 32d of yoke 32b so that the rod-receiving opening 32d of pedicle screw 32 is aligned and parallel with tubular body slot 12e of reducer 10.

With rod reducer 10 attached to pedicle screw yoke 32b, both locking element buttons 26c are squeezed to release the contact between locking elements 26 and tubular body 12, Sleeve 18 is then manually pulled distally to the second position shown in FIG. 6C. In this second position locking sleeve locking members 18d overlap anchor members 12d, radially constraining movement of anchor members 12d and providing a secure attachment between anchor members 12d and pedicle screw 32 by maintaining anchor members 12d in engagement with yoke groove 32d. The secure attachment in the second position is maintained as latch contact portions 26b reside in cavities 12L (as shown in FIG. 2) under the bias provided by torsion spring 30.

At this point, the desired amount of rod reduction can be determined and set. The amount of reduction can be read through at least window 12h on one side of the reducer 10, as shown in FIG. 1. To achieve movement of the rod reduction member 14 distally and rod reduction, reduction knob 16 is turned counterclockwise until rod contacting surface 14d contacts fixation rod 34. Rotation of reduction knob 16 may be effected by attaching a suitable tool such as a wrench (not shown) to engagement portion 16a. The desired amount of reduction may be within a range of, for example, 0 and 45 mm for lumbar spine procedures. Depending upon the application, such as in other sections of the spine, other suitable reduction ranges may apply. As a result of axial movement of rod reduction member 14, one of indicia 14g spaced axially thereon will move into alignment with window 12h for display, as illustrated in FIG. 1, thereby providing a visual indication to the surgeon of the amount of reduction that would be effected. In the arrangement where a second window 12h and a second series of indicia 14g are provided, the amount of rod reduction may be read on either side of rod reducer 10.

Further rotation of reduction knob 16 counterclockwise causes rod reduction member 14 to push down distally on fixation rod 34 and pull up on yoke 32b as shown in FIG. 6E, thereby persuading fixation rod 34 towards yoke 32b and reducing rod 34 to yoke 32b and the vertebral body to which pedicle screw 32 is attached. A locking member in the form of a set screw may then be passed through tubular body lumen 12c of rod reducer 10 to provisionally lock rod 34 into pedicle screw 32. Once rod 34 is locked into yoke 32b, rod reducer 10 may be disengaged from yoke 32b by depressing the two buttons 26c on locking sleeve 18 while simultaneously sliding locking sleeve 18 towards the proximal end of reducer 10 as shown in FIG. 6F. Anchor attachment members 12b of reducer 10 will flexibly splay outwardly, allowing reducer 10 to be separated from yoke 32b as illustrated in FIG. 6G. It should be appreciated that rod reduction member 14 does not need to be retracted prior to removal of reducer 10 from pedicle screw 32. Optionally, a surgeon may choose, however, to first retract rod reduction member 14 prior to separating rod reducer 10 from pedicle screw 32 by clockwise rotation of reduction knob 16 to thereby move reduction member 14 proximally relative to tubular body 12.

Having described the construction and operation of rod reducer 10, it should be understood that there are a number of particularly desirable features. For example, in one aspect of the subject rod reducer 10, there is independent movement of the rod reduction member and the locking sleeve. This allows the rod reducer to be attached to a pedicle screw separate from the process of rod reduction either before or after the rod is inserted. This feature also allows the rod reducer to be removed from the pedicle screw after rod reduction without withdrawing the rod reduction member, thereby saving the surgeon time to complete the procedure.

In another aspect of the subject rod reducer 10, the user can determine and set how much rod reduction is desired prior to attaching the instrument.

It should therefore be understood that while various aspects of the invention have been presented herein, various changes, modifications and further applications may be made without departing from the spirit of the invention and the scope of the appended claims.

What is claimed is:

1. A spinal rod reducer for reducing a spinal rod into a rod-receiving opening of a bone anchor, comprising;
an elongate tubular body having a proximal end, a distal end and a lumen extending therethrough, and a pair of spaced flexible anchor attachment members adjacent the distal end of said tubular body, said spaced attachment members defining a slot therebetween, the distal end of said attachment members being configured to engage said bone anchor in a manner to align said slot with said rod-receiving opening of said bone anchor;
a threaded rod reduction member supported for axial movement within said lumen, said rod reduction member including a rod contacting surface movable to a position between said attachment members;
a locking sleeve supported for axial movement on said tubular body, said locking sleeve including a pair of spaced locking members defining a channel therebetween, said locking sleeve channel being aligned with said rod reduction member slot during axial movement of said locking sleeve, said locking members being configured to respectively slidably engage said attachment members during said axial movement of said locking sleeve, said locking sleeve including a locking element for releasably securing said locking sleeve in a fixed axial position relative to said tubular body attachment members;
a threaded rotatable reduction knob threadably engaged with said reduction rod member, rotation of said reduction knob causing said rod reduction member to move axially relative to said tubular body and thereby move said rod contacting surface within said slot between said attachment members, movement of said rod reduction member being independent of movement of said locking sleeve.

2. The spinal rod reducer of claim 1, wherein said reduction knob is attached to the proximal end of said tubular body within said lumen in a manner to allow rotational but not axial movement relative to said tubular body.

3. The spinal rod reduction of claim 2, wherein said reduction knob is generally cylindrical having an outer surface and an inner surface, said inner surface including threads.

4. The spinal rod reduction of claim 3, wherein said rod reduction member is generally cylindrical having a distal end and a proximal end, said rod contacting surface being disposed at said distal end, said proximal end having exterior threads in threaded engagement with the threads on the inner surface of said reduction knob.

5. The spinal reduction rod of claim 1, further comprising at least one pin supported by said tubular body, said pin extending transversely outwardly from said tubular body.

6. The spinal reduction rod of claim 5, wherein said locking sleeve has an axially extending elongated slot in receipt of said at least one pin for limiting the axial movement of said locking sleeve.

7. The spinal reduction rod of claim 6, wherein said rod reduction member has an axially extending elongated slot in receipt of said at least one pin for preventing rotational movement of said rod reduction member relative to said tubular body.

8. The spinal reduction rod of claim 1, wherein said locking element comprises a spring-loaded latch pivotally supported exteriorly on said locking sleeve, said latch having a contact portion configured to releasably engage a portion of said tubular body.

9. The spinal reduction rod of claim 8, wherein said locking element comprises a pair of said spring-loaded latches, said latches being disposed approximately oppositely on the exterior of said locking sleeve.

10. The spinal reduction rod of claim 1, wherein said tubular body has a window exposing a portion of said rod reduction member.

11. The spinal reduction rod of claim 10, wherein said rod reduction member includes thereon a series of spaced indicia that individually align with said window upon axial movement of said rod reduction member within the lumen of said tubular body, each individual indicia being representative of a distance said spinal rod is reduced into said rod-reducing opening of said bone anchor.

12. The spinal reduction rod of claim 1, wherein said reduction knob includes a tool engagement portion extending outwardly beyond the proximal end of said tubular body, the tool engagement portion being configured to mate with a tool for applying a rotational force to said reduction knob.
